(19) United States
(12) Patent Application Publication
(10) Pub. No.: US 2009/0018544 A1
(43) Pub. Date: Jan. 15, 2009

(54) METHOD AND APPARATUS FOR SOFT TISSUE BALANCING

(75) Inventor: Jackson R. Heavener, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 11/777,472

(22) Filed: Jul. 13, 2007

(51) Int. Cl. A61B 17/66

(52) U.S. Cl. 606/90

(57) ABSTRACT

A soft tissue balancer in the form of a tensioner, a controller, and software for operating the controller and, correspondingly, the tensioner. The tensioner may be configured for receipt between the femur and the tibia and includes a pair of condylar components having individual, extendable support platforms. The support platforms may be raised or lowered to contact the femoral condyles and distraction the tibia and femur for ligament tensioning and soft tissue balancing. Once the support platforms contact the femoral condyles, range of motion testing of the knee joint may be performed and the various heights achieved and/or forces experienced by the support platforms recorded.

### BACKGROUND

1. Field of the Invention

The present invention relates to soft tissue balancing.

2. Description of the Related Art

In a knee replacement procedure, the worn and/or damaged articulating surfaces of the tibia and femur forming the knee joint are replaced with prosthetic components. To determine the appropriate size and configuration of prosthetic components needed to properly replicate the knee joint of an individual patient, ligament tension and femoral/tibial spacing may be analyzed. For example, a balancer may be inserted between the tibia and the femur to distract the tibia and the femur from one another. As a result of the distraction, the ligaments of the knee joint are tightened and the corresponding spacing between the tibia and femur may be measured. Based on the tension in the ligaments, a surgeon may then determine whether the release of any of the ligaments is necessary to achieve proper soft tissue balance in the knee joint of the patient. Balancing of the soft tissue allows for the proper distraction and force distribution within the knee joint.

### SUMMARY

The present invention relates to soft tissue balancing. In one exemplary embodiment, the present invention provides a soft tissue balancer in the form of a tensioner, a controller, and software for operating the same. The tensioner may be configured for receipt between a femur and a tibia and includes a pair of condylar components having individual, extendable support platforms. The support platforms may be raised or lowered to contact the femoral condyles and distract the tibia and femur for ligament tensioning and soft tissue balancing. Once the support platforms contact the femoral condyles, range of motion testing of the knee joint may be performed and the various heights achieved and/or forces experienced by the support platforms recorded.

In one exemplary embodiment, the movement of the support platforms of the tensioner are actuated by the controller. For example, the controller may include a hydraulic reservoir and may be capable of pumping hydraulic fluid to the support platforms of the tensioner for independent or combined actuation of the support platforms. By independently controlling the movement of the support platforms of the tensioner, one of the medial and the lateral condyles of the femur may be distracted from the tibia by a first distance and other of the medial and lateral condyles may be distracted from the tibia by a second distance. Additionally, the controller may be configured to provide hydraulic fluid to the support platforms of the tensioner at a constant pressure. The knee may then be subjected to range of motion testing and the varying distraction distances achieved and forces experienced at various points throughout the testing recorded. In one exemplary embodiment, the distances achieved and forces experienced are recorded substantially continuously throughout the range of motion testing.

Advantageously, the present invention provides a surgeon with quantitative information to assist in the performance and analysis of soft tissue balancing. For example, in one embodiment, the regulation of the pressure applied to the support platforms of the tensioner by the controller allows for the distraction distances to be dynamically measured throughout the entire range of motion. From this data, a computer connected to the soft tissue balancer of the present invention and running the software of the present invention may be used to determine the variable spring constants of ligaments and tendons of the knee joint. This information may then be used to provide the surgeon with the force received on the articulating surfaces of the tibia and femur at various points throughout the range of motion.

Additionally, when the heights of the support platforms of the tensioner are maintained at fixed heights, i.e., when the tensioner is utilized as a variable spacer block, a surgeon, at any time during the procedure, may increase or decrease the height of the support platforms of the tensioner. By increasing or decreasing the height, the surgeon is instantaneously provided with the desired amount of distraction, without the need to remove and replace a fixed spacer block. Further, the present invention also provides the surgeon with the ability to quantify planar laxity, i.e., laxity of the ligaments in an anterior-posterior plane. In contrast to traditional procedures in which a surgeon moves the ligaments medially/laterally by utilizing one of the surgeon's fingers, the height of one support platform may be maintained during range of motion testing, while the pressure of fluid supplied to the other support platform is maintained during testing. Thus, the distraction distance of the pressure constant support platform may vary in response to the forces applied on the joint by the surrounding ligaments. This provides the surgeon with a quantification of the force resulting from the gross soft tissue, such as tendons and ligaments, related to the knee joint. Additionally, by recording the varying distraction distance of the pressure constant support platform during range of motion testing, the surgeon is also provided with an additional quantification of planar laxity and other measurements useful in calculating the spring constant of the surrounding ligaments.

In one form thereof, the present invention provides a method of performing soft tissue balancing of a knee joint, the knee joint including a femur having a pair of condyles and a tibia, the method including inserting a tensioner between the femur and the tibia; aligning a first condylar component of the tensioner with one of the pair of condyles of the femur and a second condylar component of the tensioner with the other of the pair of condyles of the femur, the first condylar component including a first support platform and the second condylar component including a second support platform; applying predetermined, fixed pressure to each of the first and second support platforms to extend the first support platform by a first distraction distance and the second support platform by a second distance; maintaining the predetermined, fixed pressures during each of the following steps: subjecting the knee joint to range of motion testing; and measuring the distraction distances of the first and second support platforms throughout the range of motion testing.

In another form thereof, the present invention provides a method performing soft tissue balancing of a knee joint, the knee joint including a femur having a pair of condyles and a tibia, the method including inserting a tensioner between the femur and the tibia; aligning a first condylar component of the tensioner with one of the pair of condyles of the femur and a second condylar component of the tensioner with the other of the pair of condyles of the femur, the first condylar component including a first support platform and the second condylar component including a second support platform; extending the first support platform by a predetermined, fixed distance; applying a predetermined, fixed pressure to the second support platform to extend the second support platform by a second distance; maintaining the predetermined, fixed distance and the predetermined pressure during each of the following steps: subjecting the knee joint to range of motion testing; and measuring the second distance throughout the range of motion testing.

In yet another form thereof, the present invention provides a method performing soft tissue balancing of a knee joint, the knee joint including a femur having a pair of condyles and a tibia, the method including: inserting a tensioner between the femur and the tibia; aligning a first condylar component of the tensioner with one of the pair of condyles of the femur and a second condylar component of the tensioner with the other of the pair of condyles of the femur, the first condylar component including a first support platform and the second condylar component including a second support platform; extending the first support platform by a first, fixed distance and the second support platform by a second, fixed distance; maintaining the first, fixed distance and the second, fixed distance during each of the following steps: subjecting the knee joint to range of motion testing; and measuring the pressures received by the first and second support platforms throughout the range of motion testing.

### BRIEF DESCRIPTION OF THE DRAWINGS

The above-mentioned and other features and advantages of this invention, and the manner of attaining them, will become more apparent and the invention itself will be better understood by reference to the following descriptions of embodiments of the invention taken in conjunction with the accompanying drawings, wherein:

FIG. 1 is a perspective view of a tensioner according to an exemplary embodiment of the present invention;

FIG. 2 is a perspective view of a tensioner according to another exemplary embodiment;

FIG. 3 is another perspective view of the tensioner of FIG. 2;

FIG. 4 is another perspective view of the tensioner of 2;

FIG. 5 is a schematic view of a controller according to an exemplary embodiment;

FIG. 6 is a fragmentary, anterior view of a knee joint including a tensioner positioned between the tibia and the femur;

FIG. 7 is another fragmentary, anterior view of the knee joint including a tensioner positioned between the tibia and femur;

FIG. 8 is another fragmentary, anterior view of a knee joint including a tensioner positioned between the tibia and femur; and

FIG. 9 is a fragmentary, medial view of the knee joint including a tensioner positioned between the tibia and the femur.

Corresponding reference characters indicate corresponding parts throughout the several views. The exemplifications set out herein illustrate preferred embodiments of the invention and such exemplifications are not to be construed as limiting the scope of the invention in any manner.

### DETAILED DESCRIPTION

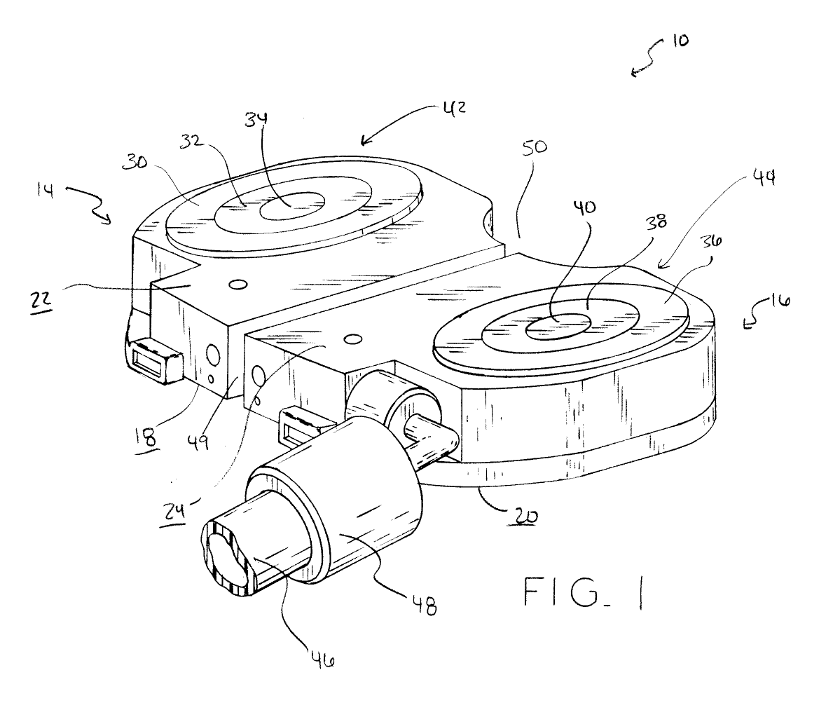

Referring to FIGS. 1 and 5, several components of the soft tissue balancer of the present embodiment, including tensioner 10 and a schematic of controller 12, are shown. Tensioner 10 may be actuated to contact condyles 84, 86 (FIG. 6) of femur 82 and to distract tibia 28 and femur 82 (FIG. 6). The actuation of tensioner 10 may be regulated by controller 12. Controller 12 may be connected to a computer operating software (not shown) made in accordance with the present invention for controlling the operation of controller 12 and recording feedback received from tensioner 10 and controller 12. Thus, with the use of the software, the forces experienced by tensioner 10 and the distraction distances achieved during range of motion testing of the knee joint may be set, monitored, and/or recorded.

Referring to FIG. 1, tensioner 10 includes condylar components 14, 16 having bottom surfaces 18, 20 and top surfaces 22, 24, respectively. Positioned within each of condylar components 14, 16 are a series of nested sections, e.g., a series of telescoping concentric cylinders 30, 32, 34 and 36, 38, 40, forming support platforms 42, 44, respectively. Support platforms 42, 44 of tensioner 10 may be raised to extend from top surfaces 22, 24, respectively, as a result of the receipt of fluid by tensioner 10 through tube 46. While tensioner 10 is described in detail herein as a utilizing a hydraulic system, tensioner 10 may utilize any known system capable of achieving the results set forth herein, including various pneumatic, mechanical, electromechanical, and electromagnetic systems. Tube 46 is connected to tensioner 10 via fluid input 48. Additionally, as described in detail below, the flow of fluid through tube 46 and into fluid input 48 of tensioner 10 may be regulated by controller 12 (FIG. 5). In another exemplary embodiment, controller 12 may be absent. In this embodiment, the flow of fluid through tube 46 and into fluid input 48 of tensioner 10 may be regulated by use of a hand pump (not shown), for example.

Fluid received by fluid input 48 may be separated within tensioner 10 by an internal mechanism (not shown) that bifurcates the fluid and regulates the pressure and/or volume thereof. In another exemplary embodiment, described in detail below, the mechanism that regulates the pressure and volume of fluid supplied to tensioner 10 is external of tensioner 10 and forms a portion of controller 12. As fluid is received within fluid input 48 and bifurcated by the internal mechanism contained within tensioner 10, fluid is directed to each of support platforms 42, 44 to begin raising concentric cylinders 30, 32, 34 and 36, 38, 40, respectively. By utilizing concentric cylinders, the overall height of tensioner 10 may be reduced. Specifically, the total height over which support platform 42 may be raised is divided amongst each of cylinders 30, 32, 34 and 36, 38, 40. As a result, support platforms 42, 44 can reach a combined height substantially greater than the height of the individual cylinders.

In addition to support platforms 42, 44, tensioner 10 includes gap 49 formed between condylar components 14, 16. By increasing or decreasing the size of gap 49, the separation of condylar components 14, 16 may be varied. Advantageously, by providing variability to the distance between condylar components 14, 16, tensioner 10 may be utilized with varying patient anatomies and adjusted to align support platforms 42, 44 with respective condyles of a patient's femur, as shown in FIG. 6. Additionally, tensioner 10 includes void 50 formed in a posterior portion of condylar components 14, 16. Void 50 may aligned with the intercondylar notch of a femur to allow for the retention of various ligamentous and muscular structure during the balancing of a knee joint.

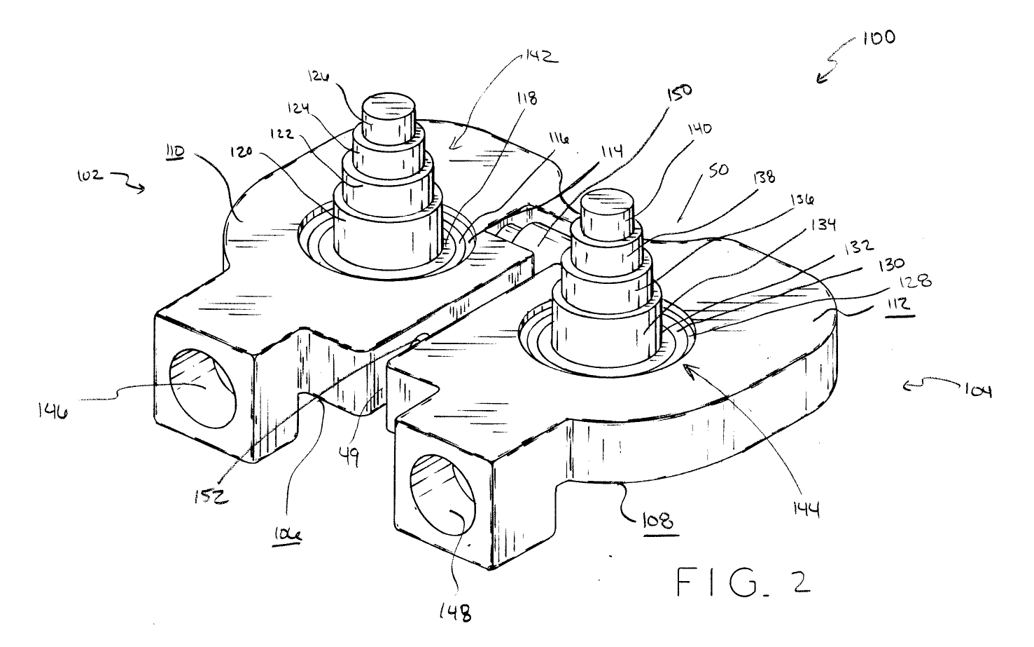

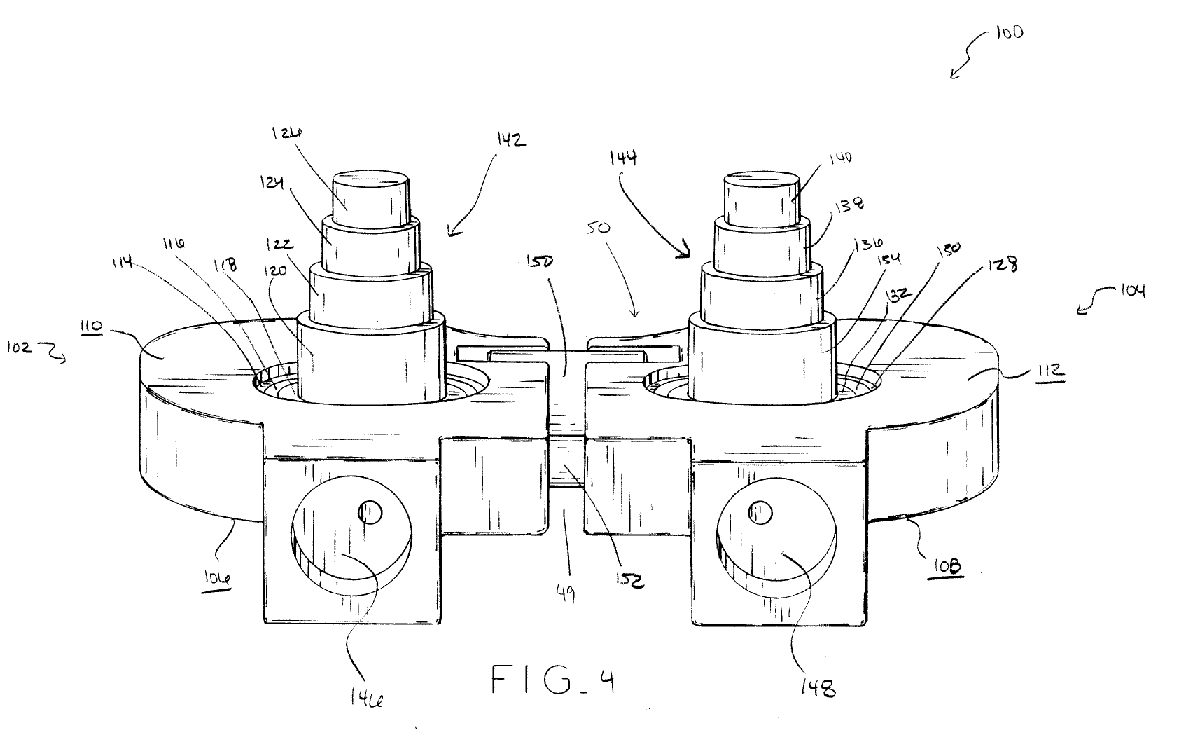

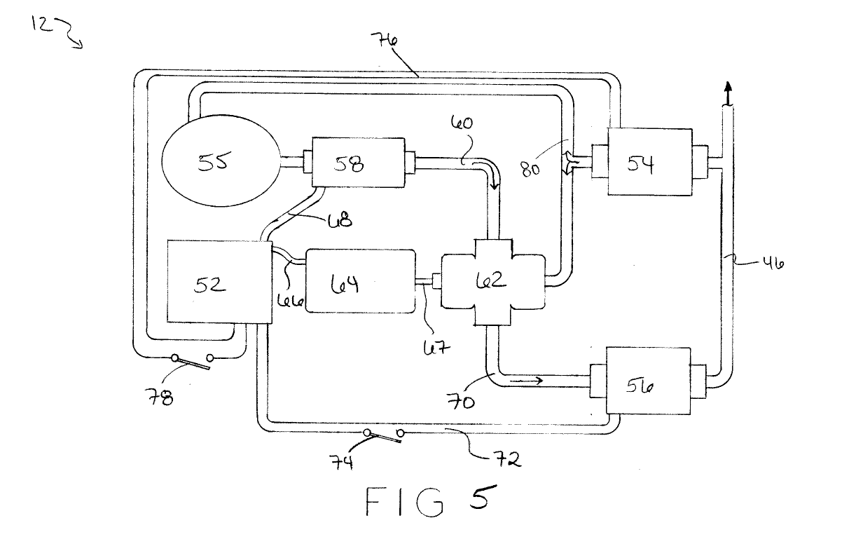

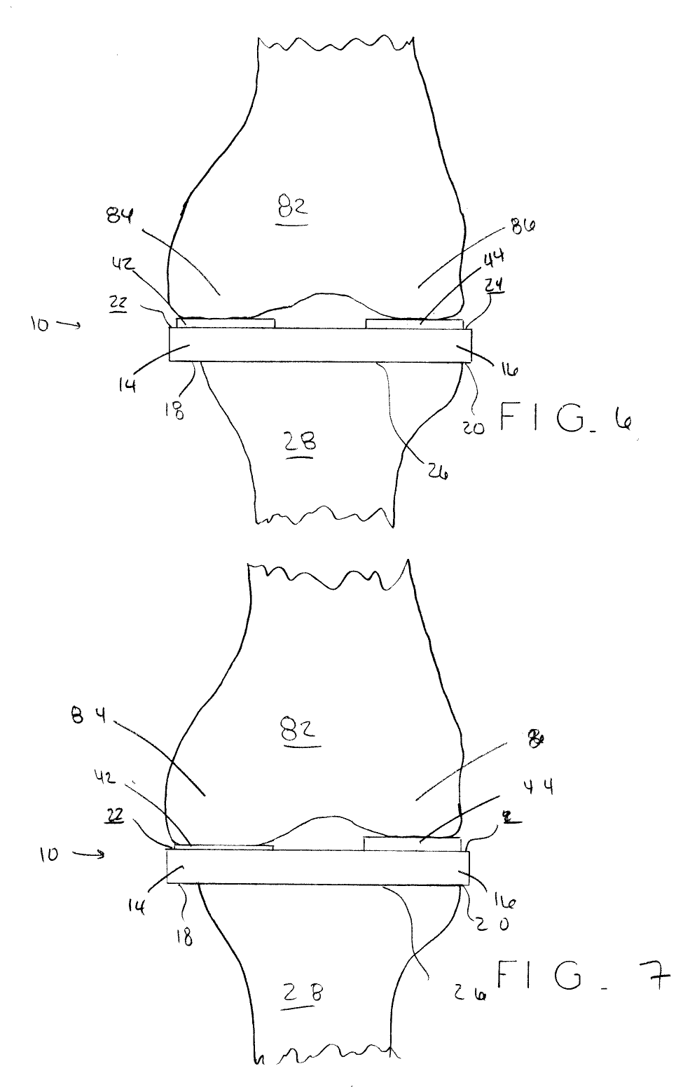

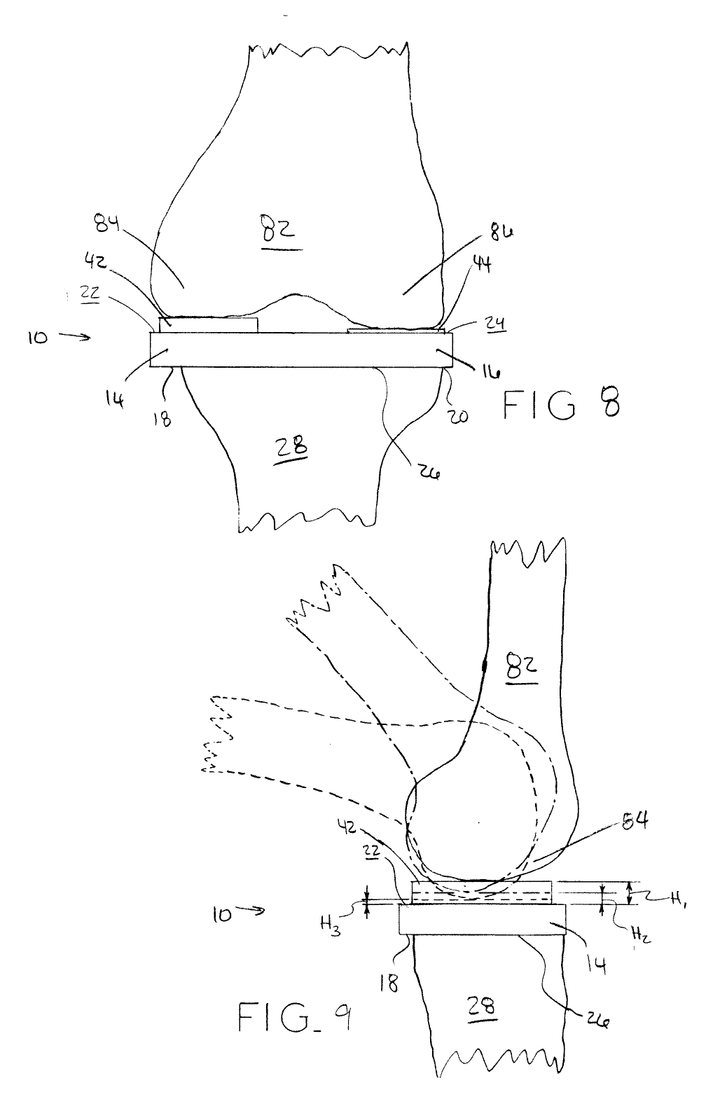

Referring to FIGS. 2-4, another exemplary embodiment of the tensioner of the present invention is depicted as tensioner 100. Tensioner 100 has several components that are identical or substantially identical to corresponding components of tensioner 10 of FIG. 1 and identical reference numerals have been used to identify identical or substantially identical components therebetween. Tensioner 100 includes condylar components 102, 104 having bottom surfaces 106, 108 and top surfaces 110, 112, respectively. Positioned within each of condylar components 102, 104 are a series of nested sections, e.g., a series of telescoping concentric cylinders 114, 116, 118, 120, 122, 124, 126 and 128, 130, 132, 134, 136, 138, 140, respectively, combining to form support platforms 142, 144, respectively. Support platforms 142, 144 of tensioner 100 function in a similar manner to support platforms 42, 44 of tensioner 10. However, by adding additional concentric cylinders, the height of tensioner 100 may be further reduced, while still allowing tensioner 100 to achieve a distraction distance substantially similar to the distraction distance that can be achieved by using tensioner 10.

Tensioner 100 further includes fluid inputs 146, 148 which may be connected to a source of fluid via tubing (not shown). The receipt of fluid by fluid inputs 146, 148 may be regulated by controller 12, as described in detail below. In another exemplary embodiment, receipt of fluid by fluid inputs 146, 148 may be regulated by a hand pump (not shown). By providing individual fluid inputs 146, 148 for condylar components 102, 104, respectively, the need for means for bifurcating the flow of fluid to the individual condylar components is eliminated. Thus, the fluid received by condylar components 102, 104 may be provided individually to condylar components 102, 104 by a single controller 12 or, in another exemplary embodiment, an individual controller 12 may be connected to each of condylar components 102, 104 via fluid inputs 146, 148, respectively. As shown in FIGS. 2-4, a substantially equal amount of fluid has been provided to condylar portions 102, 104, causing support platforms 142, 144 and, specifically, cylinders 120, 122, 124, 126 and 134, 136, 138, 140 to extend above top surfaces 110, 112 of condylar portions 102, 104, respectively, by a substantially equal distance.

As shown in FIGS. 2-4, tensioner 100 also includes connectors 150, 152 extending across gap 49 and connecting condylar components 102, 104 to one another. Connectors 150, 152 may be extended or retracted in the directions of condylar components 102, 104, allowing connectors 150, 152 to adjust the size of gap 49 between condylar components 102, 104. In one exemplary embodiment, connectors 150, 152 are hydraulic cylinders. In this embodiment, connectors 150, 152 may be connected to controller 12 via an additional fluid input (not shown). Alternatively, connectors 150, 152 may receive fluid from one or both of fluid inputs 146, 148 of condylar components 102, 104, respectively. In another exemplary embodiment, connectors 150, 152 are actuated by mechanical means, such as a detent mechanism. Irrespective of the mechanism used to actuate connectors 150, 152, connectors 150, 152 allow for condylar components 102, 104 to be adjusted, i.e., allow for the size of gap 49 to be varied, to fit an individual patient's anatomy by aligning support platforms 142, 144 with the condyles of the patient's femur, as described above.

Referring to FIG. 5, controller 12 is connectable to fluid inputs 48 and 146, 148 of tensioners 10, 100, respectively, via tube 46 and may be used to regulate the volume and/or pressure of fluid delivered to the respective condylar portions of tensioners 10, 100. Controller 12 also includes power supply 52 for controlling and regulating power to solenoid valves 54, 56, pump 58, and stepper motor 64 via electrical connections 76, 72, 68, 66, respectively. The operation of power supply 52 and, correspondingly, the operation of controller 12 may be regulated by the software of the present invention running on a computer. During operation of controller 12, fluid contained within reservoir 55 may be drawn via pump 58 through pressure feed line 60. The fluid in pressure feed line 60 is then received by an input of pressure regulator 62. Pressure regulator 62 is an adjustable pressure regulator connected to stepper motor 64 via control arm 67. By placing stepper motor 64 in electrical communication with power supply 52 via electrical connection 66, stepper motor 64 is activated and control arm 67 actuated to adjust the outlet pressure setting of pressure regulator 62. By adjusting the outlet pressure setting of pressure regulator 62, pressure regulator 62 can dynamically adjust the pressure of the fluid supplied to feed line 70.

With control arm 67 properly positioned to set the outlet pressure of pressure regulator 62 at a predetermined pressure, fluid received by pressure regulator 62 is pressurized to the predetermined pressure. The fluid then travels through the output of pressure regulator 62 and enters feed line 70. After passing through feed line 70, the fluid reaches solenoid valve 56, which is connected to power supply 52 via electrical connection 72. Positioned along electrical connection 72 is switch 74, which is in the open position during normal operation of controller 12. With switch 74 in the open position, solenoid valve 56 is correspondingly open and fluid received therein is allowed to pass therethrough to tube 46 for delivery to fluid inputs 48 and 146, 148 of tensioners 10, 100, respectively, for example. Additionally, in order to direct the fluid into fluid inputs 48 and 146, 148 of tensioners 10, 100, solenoid valve 54, which is connected to power supply 52 via electrical connection 76, is closed. Specifically, switch 78 of electrical connection 76 is maintained in the closed position, correspondingly maintaining solenoid valve 54 in the closed position during normal operation of controller 12. In contrast, if switches 74, 78 remain open, corresponding solenoid valves 56, 54, respectively, also remain open and the fluid circulates through controller 12. Specifically, fluid pumped from reservoir 55 through pressure feed line 60, pressure regulator 62, feed line 70, solenoid valve 56, and tube 46 would pass through open solenoid valve 54 and travel through return line 80 to arrive back at reservoir 55.

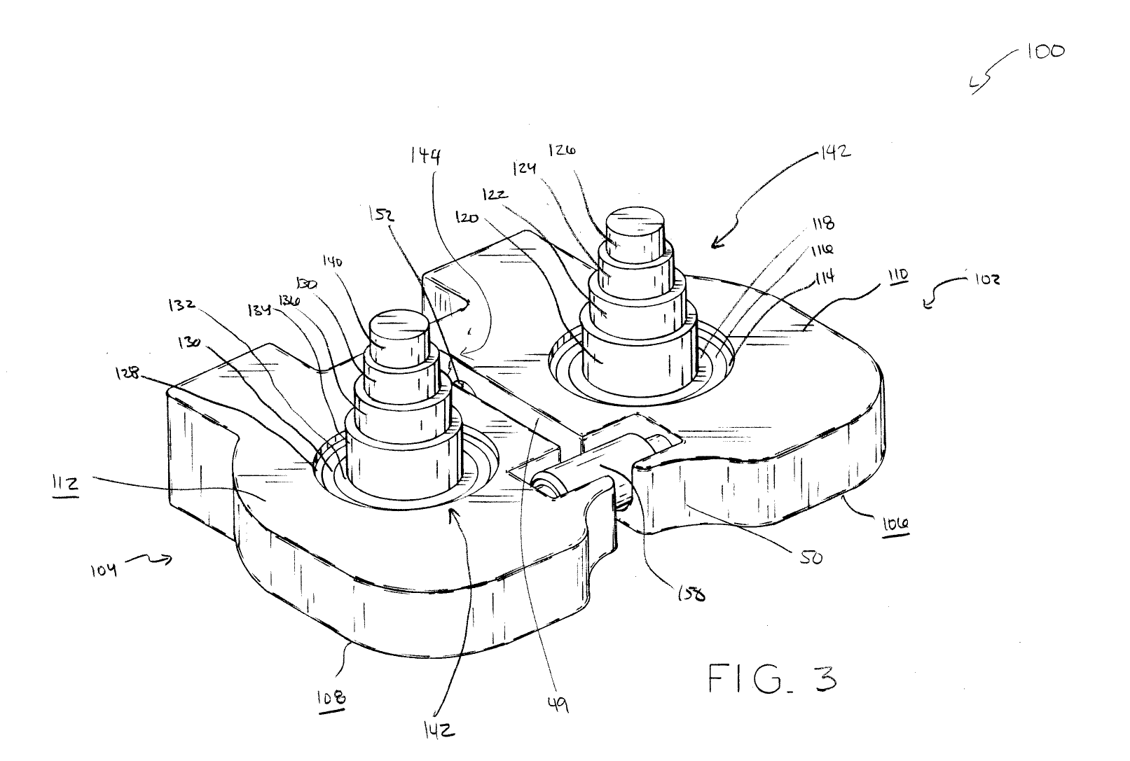

By directing fluid into fluid inputs 48 and 146, 148 of tensioners 10, 100, respectively, support platforms 42, 44 and 142, 144 are actuated to extend above top surfaces 22, 24 and 110, 112, respectively. Specifically, taking support platform 142 (FIG. 3) as an exemplary support platform, as pressurized fluid enters fluid input 146, cylinders 114, 116, 118, 120, 122, 124, and 126 may begin to rise. Cylinders 114, 116, 118, 120, 122, 124, and 126 forming support platform 142 may continue to rise until each of cylinders 114, 116, 118, 120, 122, 124, and 126 are fully extended or until the pressure of the fluid received by fluid input 146 is equalized by the pressure on support platform 142. Equalization of the pressure of the fluid received by fluid input 146 with the pressure received by support platform 142 may result from the forces applied to support platform 142 by a femoral condyle during distraction of the femur and tibia or during range of motion testing, for example.

In order to remove the pressurized fluid from tensioners 10, 100 and place support platforms 42, 44 and 142, 144 in a non-extending position, such as the position shown in FIG. 1 with respect to tensioner 10, switch 78 (FIG. 5) of controller 12 is opened and switch 74 of controller 12 is closed, causing solenoid valve 54 to open and solenoid valve 56 to close. In this manner, fluid may exit fluid inputs 48 and 146, 148 of tensioners 10, 100, respectively, through tube 46, solenoid valve 54, and return line 80 to arrive back at reservoir 55. Return line 80 is also connected to pressure regulator 62 to provide a source for monitoring outlet pressure, which ensures the proper functioning of pressure regulator 62.

In addition to regulating the pressure of the fluid received by fluid inputs 48 and 146, 148 of tensioners 10, 100, controller 12 may also be utilized to provide a predetermined volume of fluid to fluid inputs 48 and 146, 148. By providing a predetermined volume of fluid to fluid inputs 48 and 146, 148, the height of corresponding support platforms 42, 44 and 142, 144 may be regulated. Thus, in contrast to providing fluid to condylar components 14, 16 and 102, 104 at a predetermined pressure, the fixed volume of fluid received by condylar components 14, 16 and 102, 104 causes the respective cylinders of support platforms 42, 44 and 142, 144 to extend by a fixed distance above top surfaces 22, 24 and 110, 112, respectively, to set support platforms 42, 44 and 142, 144 at a fixed distraction distance, i.e., a fixed height.

Once a predetermined volume of fluid has been provided to condylar components 14, 16 and 102, 104, switches 74, 78 of controller 12 are closed, closing solenoid valves 56, 54, respectively, and preventing fluid from flowing out of fluid inputs 48 and 146, 148. In another exemplary embodiment, controller 12 is activated to close valves (not shown) positioned within tensioners 10, 100 to prevent the flow of fluid through fluid inputs 48 and 146, 148. Thus, due to the incompressibility of fluid, the height of support platforms 42, 44 and 142, 144 are maintained during range of motion testing of a knee joint, for example, as described in detail below. Additionally, in one exemplary embodiment, a relief pressure is preset for tensioners 10, 100 by controller 12 and/or the software of the present invention. In this embodiment, when the height of support platform 42, 44 is fixed and the knee joint subjected to range of motion testing, the receipt of a force sufficient to increase the pressure of the fluid within tensioners 10, 100 to a pressure in excess of the preset relief pressure triggers a pressure release, causing controller 12 to actuate the necessary components to allow for the release of fluid from tensioners 10, 100.

Referring to FIG. 6, tensioner 10 is generically shown positioned between tibia 28 and femur 82, which cooperate to form the knee joint. While operation of the tensioners of the present invention are described in detail below with specific reference to tensioner 10, tensioner 100, as well as other tensioners manufactured in accordance with the present invention, may be used in a substantially similar manner and the description of the operation of tensioner 10 set forth below is generally applicable to other tensioners in accordance with the teachings of the present invention. Additionally, other tensioners, such as those disclosed in U.S. patent application Ser. No. 10/298,634, entitled MEASUREMENT INSTRUMENT FOR USE IN ORTHOPEDIC SURGERY, filed Nov. 18, 2002, the entire disclosure of which is expressly incorporated by reference herein, may be used in accordance with the teachings set forth herein. As shown in FIGS. 6-9, tibia 28 has been resected to form a substantially planar resected end 26 upon which bottom surfaces 18, 20 of condylar components 14, 16 rest. As shown in FIG. 6, medial condyle 84 of femur 82 is positioned upon support platform 42 of condylar component 14 and lateral condyle 86 of femur 82 is positioned upon support platform 44 of condylar component 16. Support platforms 42, 44 are then raised to the same, fixed height by receiving a fixed volume of fluid, as described in detail above, in preparation for range of motion testing. Range of motion testing, e.g., movement of femur 82 relative to tibia 28 from extension to approximately 90° of flexion, is then conducted and the forces exerted on support platforms 42, 44 by condyles 84, 86 monitored.

In one exemplary embodiment, the forces exerted on support platforms 42, 44 by condyles 84, 86 are monitored by sensors positioned within condylar components 14, 16 of tensioner 10 that calculate the pressure of the fluid within condylar components 14, 16. The sensors may be connected to a computer running the software of the present invention via outputs (not show). In one exemplary embodiment, the pressures are displayed on a computer monitor. In another exemplary embodiment, the computer running the software of the present invention records the pressure at a series of predetermined points during the range of motion testing. In another exemplary embodiment, the computer running the software of the present invention records the pressure substantially continuously throughout the range of motion testing.

By recording the pressure at a series of predetermined points, e.g., at predetermined positions of tibia 28 and femur 82 relative to one another, or substantially continuously, e.g., every time that a pressure measurement is provided by the sensor, during range of motion testing, any variations in the forces exerted by tibia 28 and femur 82 may be calculated, tracked, and recorded by the computer. This information may then be used to determine the forces received by the articulating surfaces of femur 82 and tibia 28 during joint articulation. Additionally, the information may be used to determine whether sufficient ligamentous tension exists at the tested height and to assist the surgeon in the selection of the appropriate prosthetic components. In another exemplary embodiment in which the software and computer are absent, the pressures are displayed on a display attached directly to the controller.

Additionally, by fixing the height of support platforms 42, 44, i.e., fixing the distraction distance of tibia 28 and femur 82, support platforms 42, 44 and tensioner 10 function as a variable spacer block. Thus, if a surgeon determines that a second, additional height should be tested, support platforms 42, 44 may be actuated to the second height without the need to remove and replace tensioner 10. In this embodiment, controller 12 may be activated by the computer to add or remove a predetermined volume of fluid from tensioner 10 to correspondingly raise or lower, respectively, support platforms 42, 44.

Referring to FIG. 7, tensioner 10 is generically depicted with support platforms 42, 44 being maintained at a constant pressure. In one exemplary embodiment, controller 12 is set to provide fluid to fluid input 48, which then provides the fluid to support platforms 42, 44, at a constant pressure. In one exemplary embodiment, a pressure value may be entered into the software of the present invention running on a computer and the computer may send a corresponding signal to controller 12 to provide fluid to each of support platforms 42, 44 of tensioner 10 at the predetermined pressure. Tibia 28 and femur 82 may then be subjected to range of motion testing and the varying heights of support platforms 42, 44, i.e., the distraction distances of tibia 28 and femur 82, may be monitored.

To determine the heights of platforms 42, 44, tensioner 10 may include sensors (not shown) that monitor the height of support platforms 42, 44. Additionally, the sensors may take into account the thickness of tensioner 10 to determine the total distraction distance of tibia 28 and femur 82 and provide the same to the computer and/or controller 12. In one exemplary embodiment, the sensors are connected to a computer running the software of the present invention that records the distraction distances at a series of predetermined points or continuously during the range of motion testing. By recording the heights of each of support platforms 42, 44 during range of motion testing, a surgeon may review the information to determine whether additional tissue release is necessary to achieve proper distraction of tibia 28 and femur 82. In another exemplary embodiment in which the software and computer are absent, the sensors are attached directly to controller 12 and the distraction distances are displayed on a display connected to controller 12.

In another exemplary embodiment, the forces applied to each of support platforms 42, 44 may be increased and/or decreased for additional range of motion testing or during range of motion testing. By utilizing various pressures and recording the corresponding distraction distances of femur 82 and tibia 28, the variable spring constants for the ligaments and tendons of the knee may be determined. The determination of the variable spring constants of the ligaments allows a surgeon to determine the amount of force supplied by the ligaments to push the femur and tibia toward one another, i.e., the force received on the articulating surfaces of tibia 28 and femur 82.

Referring to FIG. 8, tensioner 10 is depicted positioned between tibia 28 and femur 82 with support platform 42 set to a predetermined height and support platform 44 set to a predetermined pressure. Thus, during range of motion testing of the knee joint, support platform 42 will remain fixed at the predetermined height, as described in detail above. In contrast, support platform 44 is set to fixed, predetermined pressure and, thus, controller 12 will continue to provide fluid to support platform 44 at the fixed, predetermined pressure. As a result, during range of motion testing of the knee joint, support platform 44 will increase and/or decrease in height in response to varying forces exerted by lateral condyle 86 of femur 82 and by tibia 28 on support platform 44. By determining the distraction distance of the force constant condyle, i.e., lateral condyle 86 which articulates upon support platform 44, a quantification of planar laxity in the coronal plane may be provided. Specifically, the variation in height of support platform 44, i.e., the variation in distraction distance of lateral condyle 86 of femur 82 and tibia 28, may be utilized to extrapolate the tension in the ligaments of the knee joint at various positions of articulation of the knee.

For example, referring to FIG. 9, femur 82 is depicted at various positions, including extension and various degrees of flexion. As shown in FIG. 9, medial condyle 84 is set to predetermined, fixed height H₁ and remains at height H₁ at all times during articulation of the knee joint. In contrast, support platform 44 supporting lateral condyle 86 is set to the predetermined pressure and, at extension, has a height equal to height H₁. However, due to the increased force of lateral condyle 86 pressing against support platform 44 at the point between extension and flexion, support platform 44 is depressed to height H₂. As the range of motion testing continues, additional forces are received by lateral condyle 86 at approximately 90° of flexion, which depress support platform 44 to height H₃. By recording the distraction distances of tibia 28 and femur 82, during range of motion testing, planar laxity can quantified. A surgeon may then utilize the quantification of planar laxity to assist in the selection of an implant component that substantially replicates natural articulation and ensures proper musculature and ligamentous balance.

In one exemplary embodiment, the information gained through the use of tensioners 10, 100 in the manner set forth above allows a surgeon to perform digital templating. Specifically, a digital x-ray may be taken of a patient's anatomy and stored on a computer. Using the software of the present invention, the results recorded during the testing set forth above are applied to the digital x-ray to create a predictive model. This predictive model may be used in conjunction with a library of femoral and tibial implants to allow the software to identify the appropriate femoral and tibial implant for the individual patient from the library. Additionally, in another exemplary embodiment, the predictive model may also be used in conjunction with the software of the present invention to plan resections or tissue releases in a manner that maximizes soft tissue balancing. In another exemplary embodiment, the predictive model may also be used to identify any potential soft tissue problems before any additional resections of tibia 28 or of femur 82 and/or any tissue releases have been made.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of performing soft tissue balancing of a knee joint, the knee joint including a femur having a pair of condyles and a tibia, the method comprising:
inserting a tensioner between the femur and the tibia;
aligning a first condylar component of the tensioner with one of the pair of condyles of the femur and a second condylar component of the tensioner with the other of the pair of condyles of the femur, the first condylar component including a first support platform and the second condylar component including a second support platform;
applying predetermined, fixed pressure to each of the first and second support platforms to extend the first support platform by a first distraction distance and the second support platform by a second distance;
maintaining the predetermined, fixed pressures during each of the following steps:
subjecting the knee joint to range of motion testing; and
measuring the distraction distances of the first and second support platforms throughout the range of motion testing.

2. The method of claim 1, wherein the first and second support platforms comprise a series of nested sections.

3. The method of claim 2, wherein the series of nested sections comprise a series of telescoping concentric cylinders.

4. The method of claim 1, wherein the predetermined, fixed pressure applied to the first support platform is different than the predetermined, fixed pressure applied to the second support platform.

5. The method of claim 1, wherein the predetermined, fixed pressure applied to the first support platform is substantially equal to the predetermined, fixed pressure applied to the second support platform.

6. The method of claim 1, wherein the aligning step further comprises the step of adjusting the distance between the first condylar component and the second condylar component of the tensioner.

7. The method of claim 6, wherein the adjusting step further comprises actuating a connector to alter the size of a gap between the first condylar component and the second condylar component of the tensioner.

8. A method performing soft tissue balancing of a knee joint, the knee joint including a femur having a pair of condyles and a tibia, the method comprising:
inserting a tensioner between the femur and the tibia;
aligning a first condylar component of the tensioner with one of the pair of condyles of the femur and a second condylar component of the tensioner with the other of the pair of condyles of the femur, the first condylar component including a first support platform and the second condylar component including a second support platform;
extending the first support platform by a first, fixed distance and the second support platform by a second, fixed distance;
maintaining the first, fixed distance and the second, fixed distance during each of the following steps:
subjecting the knee joint to range of motion testing; and
measuring the pressures received by the first and second support platforms throughout the range of motion testing.

9. The method of claim 8, wherein the extending step further comprises the step of supplying a fixed volume of fluid to each of the first and second support platforms.

10. The method of claim 9, wherein the fixed volume of fluid supplied to the first support platform is substantially equal to the fixed volume of fluid supplied to the second support platform.

11. The method of claim 9, wherein the fixed volume of fluid supplied to the first support platform is different than the fixed volume of fluid supplied to the second support platform.

12. The method of claim 9, wherein the first and second support platforms comprise a series of nested sections.

13. The method of claim 12, wherein the series of nested sections comprise a series of telescoping concentric cylinders.

14. A method performing soft tissue balancing of a knee joint, the knee joint including a femur having a pair of condyles and a tibia, the method comprising:
inserting a tensioner between the femur and the tibia;
aligning a first condylar component of the tensioner with one of the pair of condyles of the femur and a second condylar component of the tensioner with the other of the pair of condyles of the femur, the first condylar component including a first support platform and the second condylar component including a second support platform;
extending the first support platform by a predetermined, fixed distance;
applying a predetermined, fixed pressure to the second support platform to extend the second support platform by a second distance;
maintaining the predetermined, fixed distance and the predetermined pressure during each of the following steps:
subjecting the knee joint to range of motion testing; and
measuring the second distance throughout the range of motion testing.

15. The method of claim 14, further comprising, after the subjecting step, the additional step of measuring the force received by the first support platform throughout the range of motion testing.

16. The method of claim 14, wherein the first and second support platforms comprise a series of nested sections.

17. The method of claim 16, wherein the series of nested sections comprise a series of telescoping concentric cylinders.

18. The method of claim 14, wherein the extending step further comprises the step of supplying a fixed volume of fluid to the first support platform.
